SEQUENCE LISTING (55 sequences)

SEQ ID NO: 1 - protein, 12 aa, Artificial Sequence
  Synthetic
KNGPWYAYTGRQ

SEQ ID NO: 2 - protein, 8 aa, Artificial Sequence
  Synthetic
NWAVWXKR

SEQ ID NO: 3 - protein, 9 aa, Artificial Sequence
  Synthetic
YXXEDLRRR

SEQ ID NO: 4 - protein, 8 aa, Artificial Sequence
  Synthetic
XXPVDHGL

SEQ ID NO: 5 - protein, 9 aa, Artificial Sequence
  Synthetic
LVRSTGQFV

SEQ ID NO: 6 - protein, 9 aa, Artificial Sequence
  Synthetic
LVSPSGSWT

SEQ ID NO: 7 - protein, 9 aa, Artificial Sequence
  Synthetic
ALRPSGEWL

SEQ ID NO: 8 - protein, 9 aa, Artificial Sequence
  Synthetic
AIMASGQWL

SEQ ID NO: 9 - protein, 9 aa, Artificial Sequence
  Synthetic
QILASGRWL

SEQ ID NO: 10 - protein, 9 aa, Artificial Sequence
  Synthetic
RRPSHAMAR

SEQ ID NO: 11 - protein, 9 aa, Artificial Sequence
  Synthetic
DNNRPANSM

SEQ ID NO: 12 - protein, 9 aa, Artificial Sequence
  Synthetic
LQDRLRFAT

SEQ ID NO: 13 - protein, 9 aa, Artificial Sequence
  Synthetic
PLSGDKSST

SEQ ID NO: 14 - protein, 6 aa, Artificial Sequence
  Synthetic
FDDARL

SEQ ID NO: 15 - protein, 6 aa, Artificial Sequence
  Synthetic
FSDARL

SEQ ID NO: 16 - protein, 6 aa, Artificial Sequence
  Synthetic
FSDMRL

SEQ ID NO: 17 - protein, 6 aa, Artificial Sequence
  Synthetic
FVDVRL

SEQ ID NO: 18 - protein, 6 aa, Artificial Sequence
  Synthetic
FTDIRL

SEQ ID NO: 19 - protein, 6 aa, Artificial Sequence
  Synthetic
FNDYRL

SEQ ID NO: 20 - protein, 6 aa, Artificial Sequence
  Synthetic
FSDTRL

SEQ ID NO: 21 - protein, 6 aa, Artificial Sequence
  Synthetic
PIHYIF

SEQ ID NO: 22 - protein, 6 aa, Artificial Sequence
  Synthetic
YIHYIF

SEQ ID NO: 23 - protein, 6 aa, Artificial Sequence
  Synthetic
RIHYIF

SEQ ID NO: 24 - protein, 7 aa, Artificial Sequence
  Synthetic
IELLQAR

SEQ ID NO: 25 - protein, 10 aa, Artificial Sequence
  Synthetic
CVFXXXYXXC

SEQ ID NO: 26 - protein, 12 aa, Artificial Sequence
  Synthetic
CXFXXXYXYLMC

SEQ ID NO: 27 - protein, 13 aa, Artificial Sequence
  Synthetic
CVXYCXXXXCYVC

SEQ ID NO: 28 - protein, 13 aa, Artificial Sequence
  Synthetic
CVXYCXXXXGWXC

SEQ ID NO: 29 - protein, 8 aa, Artificial Sequence
  Synthetic
DPRATPGS

SEQ ID NO: 30 - protein, 12 aa, Artificial Sequence
  Synthetic
HLQLQPWYPQIS

SEQ ID NO: 31 - protein, 12 aa, Artificial Sequence
  Synthetic
VPWMEPAYQRFL

SEQ ID NO: 32 - protein, 12 aa, Artificial Sequence
  Synthetic
TSPLNTHNGQKL

SEQ ID NO: 33 - protein, 9 aa, Artificial Sequence
  Synthetic
RLTGGKGVG

SEQ ID NO: 34 - protein, 9 aa, Artificial Sequence
  Synthetic
CDCRGDCFC

SEQ ID NO: 35 - protein, 11 aa, Artificial Sequence
  Synthetic
ACDCRGDCGCG

SEQ ID NO: 36 - protein, 13 aa, Artificial Sequence
  Synthetic
CNGRCVSGCAGRC

SEQ ID NO: 37 - protein, 5 aa, Artificial Sequence
  Synthetic
CNGRC

SEQ ID NO: 38 - protein, 9 aa, Artificial Sequence
  Synthetic
CVCNGRMEC

SEQ ID NO: 39 - protein, 6 aa, Artificial Sequence
  Synthetic
NGRAHA

SEQ ID NO: 40 - protein, 10 aa, Artificial Sequence
  Synthetic
TAASGVRSMH

SEQ ID NO: 41 - protein, 10 aa, Artificial Sequence
  Synthetic
LTLRWVGLMS

SEQ ID NO: 42 - protein, 7 aa, Artificial Sequence
  Synthetic
CGSLVRC

SEQ ID NO: 43 - protein, 7 aa, Artificial Sequence
  Synthetic
CGLSDSC

SEQ ID NO: 44 - protein, 15 aa, Artificial Sequence
  Synthetic
NRSLKRISNKRIRRK

SEQ ID NO: 45 - protein, 15 aa, Artificial Sequence
  Synthetic
LRIKRKRRKRKKTRK

SEQ ID NO: 46 - protein, 6 aa, Artificial Sequence
  Synthetic
NRSTHI

SEQ ID NO: 47 - protein, 7 aa, Artificial Sequence
  Synthetic
SMSIARL

SEQ ID NO: 48 - protein, 7 aa, Artificial Sequence
  Synthetic
VSFLEYR

SEQ ID NO: 49 - protein, 9 aa, Artificial Sequence
  Synthetic
CPGPEGAGC

SEQ ID NO: 50 - protein, 7 aa, Artificial Sequence
  Synthetic
ATWLPPR

SEQ ID NO: 51 - protein, 6 aa, Artificial Sequence
  Synthetic
RRKRRR

SEQ ID NO: 52 - protein, 15 aa, Artificial Sequence
  Synthetic
ASSSYPLIHWRPWAR

SEQ ID NO: 53 - protein, 10 aa, Artificial Sequence
  Synthetic
CTTHWGFTLC

SEQ ID NO: 54 - protein, 34 aa, Artificial Sequence
  Synthetic
AKVKDEPQRRSARLSAKPAPPKPEPKPKKAPAKK

SEQ ID NO: 55 - protein, 34 aa, Artificial Sequence
  Synthetic
AVKPKAARALPSQRSRPPEKAKKPPDKPAPEKKK

(19) United States
(12) Patent Application Publication
(10) Pub. No.: US 2010/0098637 A1
(43) Pub. Date: Apr. 22, 2010

(54) DYE-LOADED NANOPARTICLE

(75) Inventors: Daniel Orringer, Ann Arbor, MI (US); Oren Sagher, Ann Arbor, MI (US); Raoul Kopelman, Ann Arbor, MI (US); Yong-Eun Koo Lee, Ann Arbor, MI (US)

Correspondence Address: Casimir Jones, S.C., 2275 DEMING WAY, SUITE 310, MIDDLETON, WI, 53562, US

(73) Assignee: The Regents Of The University Of Michigan, Ann Arbor, MI (US)

(21) Appl. No.: 12/565,583

(22) Filed: Sep. 23, 2009

Related U.S. Application Data
(60) Provisional application No. 61/099,348, filed on Sep. 23, 2008.

(51) Int. Cl. A61K 49/00

(52) U.S. Cl. 424/9.1; 977/773

(57) ABSTRACT

The present invention generally relates to dye-loaded nanoparticles. In particular, the present invention provides methods for the staining and visualization of tumor and tumor boundaries using dye-loaded nanoparticles.

### CROSS REFERENCE TO RELATED APPLICATIONS

The present application claims priority to U.S. Provisional Application Ser. No. 61/099,348 filed Sep. 23, 2008, herein incorporated by reference in its entirety.

### FIELD OF THE INVENTION

The present invention generally relates to dye-loaded nanoparticles. In particular, the present invention provides methods for the staining and visualization of tumor and tumor boundaries using dye-loaded nanoparticles.

### BACKGROUND OF THE INVENTION

The incidence of primary brain tumors in the United States has been estimated at approximately 43,800 per year (Statistical Report: Primary Brain Tumors in the United States, 1998-2002. Published by the Central Brain Tumor Registry of the United States, (2005), ACS. Cancer Facts & Figures 2004, Published by American Cancer Society (2004), herein incorporated by reference in their entireties). While incidence rates of cancer in general have fallen or been stable, the age-adjusted incidence of primary brain tumors has been increasing at an alarming rate over the past several decades (Gavrilovic et al., J. Neuro-Oncol., 75(1), 5-14 (2005), Brandes, Semin. Oncol., 30(6), 1-3 (2003), herein incorporated by reference in their entireties). Moreover, improvements in surgical and adjuvant therapy for brain tumors have not translated into a meaningful improvement in patient outcome (Ullrich et al., Neurol. Clin. 21(4), 897-913 (2003), herein incorporated by reference in its entirety). What are needed are improved methods for treatment of tumors.

### SUMMARY OF THE INVENTION

In some embodiments, the present invention provides a method for delineation of tumor boundaries, comprising providing a nanoparticle-based optical dye and a subject with tumor, administering the nanoparticle-based optical dye to the subject, localizing of the nanoparticle-based optical dye in the tumor, and observing the nanoparticle-based optical dye localized in the tumor. In some embodiments, the tumor is resected using the optical dye to select the boundaries of the resection (e.g., resecting all tissue that contains the dye; resecting a margin of tissue surrounding and encompassing all tissue containing the dye). In some embodiments, the nanoparticle-based optical dye is targeted to said tumor (e.g., using tumor-specific targeting moieties). In some embodiments, the nanoparticle comprises polyacrylamide. In some embodiments, the optical dye is one or more of Coomassie Blue, Methylene Blue, and/or Indocyanine Green. In some embodiments, the tumor is a brain tumor. In some embodiments, the subject is a human subject.

In some embodiments, the present invention provides a method for delineation of tumor boundaries, comprising: (a) administering a nanoparticle-based optical dye to a subject, wherein the subject has a tumor, (b) localizing of the nanoparticle-based optical dye in the tumor, and (c) observing the nanoparticle-based optical dye localized in the tumor. In some embodiments, the method for delineation of tumor boundaries further comprises the step of (d) resecting the tumor using the optical dye to select resection boundaries. In some embodiments, the nanoparticle-based optical dye is targeted to the tumor with a tumor-specific targeting moiety. In some embodiments, the tumor-specific targeting moiety is a tumor-specific peptide. In some embodiments, the tumor-specific targeting peptide comprises F3. In some embodiments, the nanoparticle comprises polyacrylamide. In some embodiments, the optical dye is selected from the group of Coomassie Blue, Methylene Blue, and or Indocyanine Green. In some embodiments, the tumor arises from cancer, selected from the list of brain cancer, bladder cancer, melanoma, breast cancer, colon cancer, rectal cancer, pancreatic cancer, endometrial cancer, prostate cancer, kidney (renal cell) cancer, skin cancer (nonmelanoma), thyroid cancer, and lung cancer. In some embodiments, the subject is a human subject. In some embodiments, the subject is a mammal, non-human primate, rodent, canine, etc. In some embodiments, the nanoparticle-based optical dye remains localized within the tumor for at least 2 hours (e.g. 2 hours, 3 hours, 4 hours, 6 hours, 8 hours, 12 hours, etc.). In some embodiments, the delineation is observable with the naked eye. In some embodiments, the present invention provides a nanoparticle-based optical dye composition comprising a nanoparticle component and an optical dye component, wherein the optical dye component is loaded into the nanoparticle component. In some embodiments, the nanoparticle component comprises polyacrylamide. In some embodiments, the nanoparticle component is selected from the group of Coomassie Blue, Methylene Blue, and Indocyanine Green. In some embodiments, the nanoparticle-based optical dye composition further comprises a tumor-specific targeting moiety. In some embodiments, the tumor-specific targeting moiety comprises a tumor-specific peptide. In some embodiments, the tumor-specific targeting peptide comprises F3. In some embodiments, the tumor-specific targeting peptide comprises Chlorotoxin. In some embodiments, the nanoparticle-based optical dye composition is stable for at least 2 hours under physiological conditions (e.g. 2 hours . . . 3 hours . . . 4 hours . . . 6 hours . . . 8 hours . . . 12 hours . . . 48 hours . . . etc.).

### BRIEF DESCRIPTION OF THE DRAWINGS

The foregoing summary and detailed description is better understood when read in conjunction with the accompanying drawings which are included by way of example and not by way of limitation.

FIG. 1 shows delineation of cancerous and non-cancerous brain tissue: administration to brain tumor model of A) free dye for 0 minutes, B) free dye for 60 minutes, C) dye-loaded nanoparticles for 0 minutes, D) dye-loaded nanoparticles for 60 minutes, and E) spectra of free dye and dye-loaded nanoparticles administered to tumor model for 0-120 minutes (Note: the differences in the curves is likely due to photobleaching).

FIG. 2 shows near-IR delineation of cancerous and non-cancerous brain tissue: A) photo of free dye at 0 minutes after administration, B) near-IR of free dye at 0 minutes after administration, C) near-IR of free dye at 60 minutes after administration, D) photo of dye-loaded nanoparticles at 0 minutes after administration, E) near-IR of dye-loaded nanoparticles at 0 minutes after administration, F) near-IR of dye-loaded nanoparticles at 60 minutes after administration, and G) spectra of free dye and dye-loaded nanoparticles administered to tumor model for 0-120 minutes.

FIG. 3 shows co-localization of GFP-expressing tumor and nanoparticles that contain rhodamine: A) tumor, B) nanoparticles, C) co-localization, and D) angiogenic vessels.

FIG. 4 shows a graphical comparison of nanoparticle staining of tumor and non-tumor tissues.

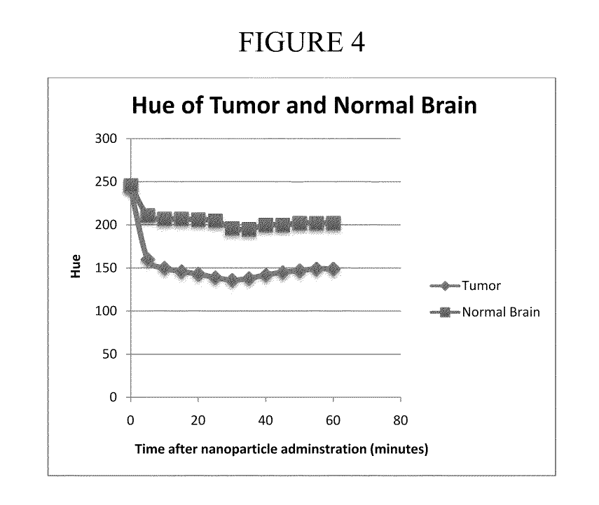

### DETAILED DESCRIPTION OF EMBODIMENTS

The incidence of primary brain tumors in the United States has been estimated at approximately 43,800 per year (Statistical Report: Primary Brain Tumors in the United States, 1998-2002. Published by the Central Brain Tumor Registry of the United States, (2005), ACS. Cancer Facts & Figures 2004, Published by American Cancer Society (2004), herein incorporated by reference in their entireties). The most common therapy given to such victims is surgical resection. The normal-tumor boundaries for different primary and secondary brain tumors vary from fingerlike protrusions of tumor cells into normal tissues in glioblastoma multiforme to well-circumscribed nodules with possible surrounding edema in most secondary tumors. The most common initial therapy for primary and secondary brain tumors is surgical resection. Many studies have shown that the degree of resection significantly influences the time to recurrence and the overall survival of brain tumor patients. Successful resection relies on complete removal of the tumor. Aggressive surgery in the brain is not acceptable, yet residual tumorous tissue left behind after resection is believed to be the main cause of morbidity. Currently, surgical navigation systems and ultrasonography are used intraoperatively to help neurosurgeons locate brain tumor and maximize resection. Surgical navigation systems enable neurosurgeons to relate the position of a surgical instrument to structures present in preoperative computerized tomography (CT) or magnetic resonance (MR) images. However, CT or MR imaging may not delineate the exact brain tumor borders. Studies have shown that neoplastic cells can be found in brain tissue outside the apparent tumor margins defined by contrast-enhanced CT or MR imaging. More importantly, the accuracy of surgical navigation systems can be degraded by registration error and intraoperative brain deformation which may shift brain tumor borders in image space by more than a centimeter from their actual locations. Ultrasonography is able to detect brain tumors because of their hyperechoic characteristics. However, peritumoral edema is also hyperechoic, which hampers tumor and tumor margin identification. Thus, despite the applications of these technologies in neurosurgery, significant residual tumor mass is often found to be left behind in patients after craniotomy. Neurosurgeons also rely on visual inspection and/or on-site pathology to locate tumors and tumor margins. Visual inspection is subjective and often incorrect as the visual characteristics of many brain tumors mimic that of normal brain. In addition, on-site pathology is expensive and time-consuming. Hence, there is a need for an objective, intraoperative real-time system which is capable of accurately differentiating brain tumors from normal brain tissue, thus detecting tumor margins.

In some embodiments, the present invention provides compositions and methods for delineation of tumor boundaries utilizing nanoparticle-based optical dyes. Experiments conducted during the development of embodiments of the present invention demonstrate that appropriately administered optical dyes permit visual inspection of tumor margins, providing guidance for more complete resection of tissue, while retaining undiseased tissue.

In some embodiments of the present invention, a nanoparticle-based optical dye is administered to a patient prior to surgical resection of a tumor (e.g., brain tumor). In some embodiments, the nanoparticle-based optical dye targets the tumor tissue, localizing the optical dye within the tumor boundaries, providing real-time, visual, distinction between the brain and tumor tissue. In some embodiments, the present invention provides surgeons with the ability to more accurately perform surgical resection.

In some embodiments, the nanoparticle comprises polyacrylamide, although a variety of nanoparticle materials can be used. In some embodiments, nanoparticles are formed from components including, but not limited to, silica, polyacrylamide, N-(3-Aminopropyl)methacrylamide), methylmethacrylates, alkylcyano-acrylates, hydroxyethylmethacrylates, methacrylic acid, ethylene glycol dimethacrylate, acrylamide, N,N′-bismethylene acrylamide, 2-dimethylaminoethyl methacrylate, N,N-L-lysinediyltere-phthalate, polylactic acid, polylactic acid-polyglycolic acid-copolymers, polyanhydrates, poly-orthoesters, gelatin, albumin, desolvated macromolecules or carbohydrates, polystyrene, poly(vinylpyridine), polyacroleine, polyglutaraldehyde, organically modified silicate, phenyltrimethoxysilane, methyltrimethoxysilane, tetraethyl orthosilicate, (3-aminopropyl)triethoxysilane, poly n-butyl acrylate: n-butyl acrylate, hexanedioldiacrylate, poly(vinyl chloride), poly(decyl methacrylate), decyl methacrylate, hexanediol dimethacrylate, poly(ethylene glycol), poly(ethylene glycol) (n)-monomethacrylate, tetraethylene glycol dimethacrylate, and fluorinated derivatives of the above.

In some embodiments, the present invention comprises an optical dye. In some embodiments, the optical dye is one or more of bromophenol blue, Coomassie Blue (e.g., Coomassie Blue R-250, G-250, etc.), Methylene Blue (3,7-bis(Dimethylamino)-phenazathionium chloride Tetramethylthionine chloride), and/or Indocyanine Green. In some embodiments, the optical dye is selected from the group including, but not limited to acridine dyes, anthraquinone dyes, arylmethan dyes, azo dyes, cyanine dyes, diazonium dyes, nitro dyes, nitroso dyes, phenaanthridine dyes, pthalocyanine dyes, quinine-imine dyes, indamins, indophenols dyes, oxazin dyes, oxazone dyes, thiazin dyes, thiazole dyes, xanthenes dyes, fluorene dyes, pyronin dyes, fluorine dyes, rhodamine dyes, any non-toxic dye, etc.

In some embodiments, the nanoparticle-based optical dye is targeted to said tumor. In some embodiments, the nanoparticle-based optical dye of the present invention provides a targeting moiety. In some embodiments, the targeting moiety is configured to target the dye to a specific particular location, tumor marker, cancer marker, cell type, diseased tissue, tumor type, cellular location, tissue, etc. In some embodiments, the targeting moiety is specific to the tissue type, tumor type, cancer type, or tumor class being treated. In some embodiments, the targeting moiety is directed against a target molecule. In some embodiments, the targeting moiety may include, for example antibodies, cell surface receptor ligands and hormones, lipids, sugars and dextrans, alcohols, bile acids, fatty acids, amino acids, peptides (e.g. tumor-specific peptides), nucleic acids, etc. In some embodiments, targeting moieties include, but are not limited to tumor-specific peptides such as F3 peptide, TAT peptide, RGD (Arg-Gly-Asp) sequence, chlorotoxin, etc. In some embodiments, targeting moieties include, but are not limited to tumor-specific small molecules such as folate or other chemotherapeutics. In some embodiments, tumor targeting moieties are bioactive peptides such as octreotate and bombesin. Contemplated biological targets include, but are not limited to, cell surface proteins, cell surface receptors, cell surface polysaccharides, extracellular matrix proteins, intracellular proteins and intracellular nucleic acids, and the like. The following table provides a listing of exemplary peptide sequences useful in targeting of tumors, tumor vasculature, and specific tumor types in accordance with the present invention.

Cancer Cell Surface or Cancer-Related Targeting
Peptides Identified by Phage-Display Library

| Peptide Ligand | Cellular Target | Ref. |
|---|---|---|
| Tumor cell | | |
| surface | | |
| KNGPWYAYTGRO | Surface idiotype | Renschler et al., |
| (SEQ ID NO: 1) | of SUP-88 human | 1994 |
| | B-cell lymphoma | |
| NWAVWXKR, | | |
| (SEQ ID NO: 2) | | |
| YXXEDLRRR | | |
| (SEQ ID NO: 3) | | |
| XXPVDHGL | | |
| (SEQ ID NO: 4) | | |
| LVRSTGQFV, | Surface idiotype | Buhl et al., 2002 |
| (SEQ ID NO: 5) | of human chronic | |
| LVSPSGSWT | lymphocytic | |
| (SEQ ID NO: 6) | lymphoma (CLL) | |
| ALRPSGEWL | | |
| (SEQ ID NO: 7) | | |
| AIMASGQWL | | |
| (SEQ ID NO: 8) | | |
| QILASGRWL, | | |
| (SEQ ID NO: 9) | | |
| RRPSHAMAR | | |
| (SEQ ID NO: 10) | | |
| DNNRPANSM, | | |
| (SEQ ID NO: 11) | | |
| LQDRLRFAT | | |
| (SEQ ID NO: 12) | | |
| PLSGDKSST | | |
| (SEQ ID NO: 13) | | |
| FDDARL | Human multiple | Szecsi et al., |
| (SEQ ID NO: 14) | myeloma M- | 1999 |
| | protein | |
| FSDARL, | | |
| (SEQ ID NO: 15) | | |
| FSDMRL | | |
| (SEQ ID NO: 16) | | |
| FVDVRL, | | |
| (SEQ ID NO: 17) | | |
| FTDIRL, | | |
| (SEQ ID NO: 18) | | |
| FNDYRL | | |
| (SEQ ID NO: 19) | | |
| FSDTRL, | | |
| (SEQ ID NO: 20) | | |
| PIHYIF, | | |
| (SEQ ID NO: 21) | | |
| YIHYIF. | | |
| (SEQ ID NO: 22) | | |
| RIHYIF | | |
| (SEQ ID NO: 23) | | |
| IELLQAR | HL 60 human | Fukuda at al., |
| (SEQ ID NO: 24) | lymphoma & B-16 | 2000 |
| | mouse melanoma | |
| CVFXXXYXXC | Prostate- | Wu et al., 2000 |
| (SEQ ID NO: 25) | specific | |
| | antigen (PSA) | |
| CXFXXXYXYLMC | | |
| (SEQ ID NO: 26) | | |
| CVXYCXXXXCYVC | | |
| (SEQ ID NO: 27) | | |
| CVKYCXXXXGWXC | | |
| (SEQ ID NO: 28) | | |
| DPRATPGS | LNCaP prostate | Romanov et al., |
| (SEQ ID NO: 29) | cancer | 2001 |
| HLQLQPWYPQIS | WAG-2 human | Zhang et al., |
| (SEQ ID NO: 30) | neuroblastoma | 2001 |
| VPWMEPAYQRFL | MDA-MB435 breast | Zhang et al., |
| (SEQ ID NO: 31) | cancer | 2001 |
| TSPLNTHNGQKL | Head and neck | Hong and Clayman, |
| (SEQ ID NO: 32) | cancer lines | 2000 |
| OSPL W/F, R/K, | ECV304 endothe- | Ivanenko et al., |
| N/H, S, V/H, L | lial | 1999 |
| | cell line | |
| RLTGGKGVG | Hep-2 human | Ivanenko et al., |
| (SEQ ID NO: 33) | larygeal | 1999 |
| | carcinoma | |
| Tumor | | |
| vasculature | | |
| CDCRGDCFC | avB3, avB5 | Koivunen et al., |
| (RGD-4C) | | 1995 |
| (SEQ ID NO: 34) | | |
| ACDCRGDCGCG | avB5, avB3 | Assa-Munt et al., |
| (SEQ ID NO: 35) | | 2001 |
| CNGRCVSGCAGRC | Aminopeptidase N | Pasqualini et al., |
| (SEQ ID NO: 36) | | 2000 |
| CNGRC | Aminopeptidase N | |
| (SEQ ID NO: 37) | | |
| CVCNGRMEC, | | |
| (SEQ ID NO: 38) | | |
| NGRAHA | | |
| (SEQ ID NO: 39) | | |
| TAASGVRSMH, | NG2 proteoglycan | Burg et al., 1999 |
| (SEQ ID NO: 40) | | |
| LTLRWVGLMS | | |
| (SEQ ID NO: 41) | | |
| CGSLVRC, | Vasculature of | Arap et al., 1998 |
| (SEQ ID NO: 42) | various tumors | |
| CGLSDSC | | |
| (SEQ ID NO: 43) | | |
| NRSLKRISNKRIRRK, | IC-12 rat | Kenel et al., 2000 |
| (SEQ ID NO: 44) | trachea | |
| LRIKRKRRKRKKTRK, | | |
| (SEQ ID NO: 45) | | |
| NRSTHI | | |
| (SEQ ID NO: 46) | | |
| SMSIARL | Mice prostate | Arap et al., 2002 |
| (SEQ ID NO: 47) | | |
| VSFLEYR | | |
| (SEQ ID NO: 48) | | |
| CPGPEGAGC | Aminopeptidase P | Essler et al., |
| (SEQ ID NO: 49) | | 2002 |
| ATWLPPR | VEGF | Binetruy-Tournaire |
| (SEQ ID NO: 50) | | et al., 2000 |
| RRKRRR | VEGF | Bae et al., 2000 |
| (SEQ ID NO: 51) | | |
| ASSSYPLIHWRPWAR | VEGF | Asai et al., 2002 |
| (SEQ ID NO: 52) | | |
| CTTHWGFTLC | Gelantinase | Koivunen et al., |
| (SEQ ID NO: 53) | | 1999 |

In some embodiments, the subject is a human subject. In some embodiments, the human subject of the present invention suffers from cancer. In some embodiments, the present invention finds use in the treatment of tumors arising from cancers, including, but not limited to bladder cancer, melanoma, breast cancer, colon cancer, rectal cancer, pancreatic cancer, endometrial cancer, prostate cancer, kidney (renal cell) cancer, skin cancer (nonmelanoma), thyroid cancer, and lung cancer.

In some embodiments, the present invention finds use in the treatment tumors. In some embodiments, the tumor is a brain tumor. In some embodiments, the present invention finds use in the treatment brain tumors. In some embodiments, brain tumors may include, but are not limited to acoustic neuroma, astrocytoma, chordoma, CNS lymphoma, craniopharyngioma, brain stem glioma, ependymoma, mixed glioma, optic nerve glioma, subependymoma, medulloblastoma, meningioma, metastatic brain tumors, oligodendroglioma, pituitary Tumors, Primitive Neuroectodermal (PNET), and Schwannoma.

In some embodiments, the present invention is practiced by administering a nanoparticle-based optical dye of the present invention to a subject. Known methods for administering therapeutics and diagnostics can be used to administer nanoparticle-based optical dyes for practicing the present invention. For example, fluids that include pharmaceutically and physiologically acceptable fluids, including water, physiological saline, balanced salt solutions, buffers, aqueous dextrose, glycerol or the like as a vehicle, can be administered by any method used by those skilled in the art. These solutions are typically sterile and generally free of undesirable matter. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected.

The invention further provides formulations comprising the nanoparticle-based optical dye of the invention and a pharmaceutically acceptable excipient, wherein the nanoparticle-based optical dye is formed according to any of the above described embodiments, and wherein the formulation is suitable for administration as an imaging enhancing agent and the nanoparticle-based optical dye is present in an amount sufficient to enhance a magnetic resonance tomography image. These agents can be administered by any means in any appropriate formulation. Detergents can also be used to stabilize the composition or the increase or decrease the absorption of the composition. Other physiologically acceptable compounds include wetting agents, emulsifying agents, dispersing agents or preservatives that are particularly useful for preventing the growth or action of microorganisms. One skilled in the art would appreciate that the choice of an acceptable carrier, including a physiologically acceptable compound depends, e.g. on the route of administration and on the particular physio-chemical characteristics of any co-administered agent.

The compositions may be administered by any convenient route, for example by infusion or bolus injection and may be administered together with other biologically active agents. Administration can be systemic or local. In addition, the nanoparticle-based optical dye(s) compositions may be introduced into the central nervous system by any suitable route, including intraventricular and intrathecal injection; intraventricular injection may be facilitated by an intraventricular catheter, for example, attached to a reservoir, such as an Ommaya reservoir.

The compositions of the invention can be delivered by any means known in the art systematically (e.g. intra-venously), regionally or locally (e.g. intra- or peri-tumoral or intra-cystic injection, e.g. to image bladder cancer) by e.g. intra-arterial, intra-tumoral, intra-venous (iv), parenteral, intra-pneural cavity, topical, oral or local administration, as sub-cutaneous intra-zacheral (e.g. by aerosol) or transmucosal (e.g. voccal, bladder, vaginal, uterine, rectal, nasal, mucosal), intra-tumoral (e.g. transdermal application or local injection). For example, intra-arterial injections can be used to have a “regional effect”, e.g. to focus on a specific organ (e.g. brain, liver, spleen, lungs). For example intra-hepatic artery injection or intra-carotid artery injection may be used. If it is decided to deliver the preparation to the brain, it can be injected into a carotid artery or an artery of the carotid system of arteries (e.g. ocipital artery, auricular artery, temporal artery, cerebral artery, maxillary artery etc.). The present invention also provides pharmaceutical compositions which include nanoparticle-based optical dye, alone or with a pharmaceutically acceptable carrier.

In some embodiments, amounts of the nanoparticle-based optical dye sufficient to provide the desired results will be used, balanced by other considerations such as whether the nanoparticle-based optical dye used for a particular application might produce undesirable physiological results. In some embodiments, the precise dose to be employed in the formulation can also depend on the route of administration, and should be decided according to the judgment of the practitioner and each subject's circumstances. In some embodiments, the amounts of the nanoparticle-based optical dye administered can range from micromolar to molar amounts, but more likely will be used in millimolar-to-micromolar amounts.

The formulations of the invention can be administered in a variety of unit dosage forms, depending upon the particular cell or tissue or cancer to be imaged, the general medical condition of each patient, the method of administration, and the like. Details on dosages are well described on the scientific and patent literature. The exact amount and concentration nanoparticle-based optical dye or pharmaceutical of the invention and the amount of formulation in a given dose, or the “effective dose” can be routinely determined by, e.g. the clinician. The “dosing regimen” will depend upon a variety of factors, e.g. whether the cell or tissue or tumor to be imaged is disseminated or local, the general state of the patient's health, age and the like. Using guidelines describing alternative dosing regimens, e.g. from the use of other imaging nanoparticle-based optical dyes, the skilled artisan can determine by routine trials optimal effective concentrations of pharmaceutical compositions of the invention.

In some embodiments compositions of the present invention, alone or in combination with other compositions, may be provided in the form of a kit. For example, targeted, dye-loaded nanoparticles may be provided in a kit for localization within and delineation of a specific tumor type. The kit may include any and all components necessary or sufficient administration to a subject, including but not limited to, crystalline powdered dye-loaded nanoparticles in sterile glass container, diluents (e.g. normal saline for dissolving powder into a solution for administration), syringes with attachment for mixing, administration tubing set with filter permeable to single nanoparticles to prevent clumping, written and/or pictorial instructions and product information, packaging, and the like. In some embodiments, the kits provide a sub-set of the required components, wherein it is expected that the user will supply the remaining components. In some embodiments, the kits comprise two or more separate containers wherein each container houses a subset of the components to be delivered.

In some embodiments, nanoparticles of the present invention will be administered at a dose of approximately 1-1000 mg of nanoparticles/kg of subject weight (e.g. 1 mg/kg . . . 5 mg/kg . . . 10 mg/kg . . . 50 mg/kg . . . 100 mg/kg . . . 500 mg/kg . . . 1.000 mg/kg). In some embodiments, the user (e.g. clinician) will determine the appropriate number of vials of crystalline nanoparticle needed to achieve color change based on a subject's (e.g. patient) weight. In some embodiments, a solution of the crystalline nanoparticles will be prepared in diluent (e.g. saline) to a concentration appropriate for administration (e.g. 10 mg/ml . . . 20 mg/ml . . . 30 mg/ml . . . 40 mg/ml . . . 50 mg/ml . . . 60 mg/ml . . . 70 mg/ml . . . 80 mg/ml . . . 90 mg/ml . . . 100 mg/ml). In some embodiments, for example, for a human patient with average body weight, at a dose of 5-500 mg NP/kg, 3.5-35g of NP will likely be required to achieve maximal color change in the tumor; the volume required for administration of 3.5-35 mg NP is (0.05 g/ml) 70-700 ml.

In addition to optical dyes, in some embodiments, the nanoparticles of the present invention further comprise additional functional agents. In some embodiments, these additional functional agents may find use in delivering photodynamic therapy (PDT), as a magnetic resonance imaging contrast agent (e.g. gadolinium containing molecule or complex), as a fluorescent dye, as therapeutics (e.g. chemotherapeutics), and the like.

In some embodiments, to address the inability of current surgical techniques to reliably eradicate residual or unresectable tumor, the present invention may be configured to deliver photodynamic therapy, in addition to optical dyes. PDT relies on activation of a photosensitizer which, when activated by a specific wavelength of light, induces the release of energy to tissue oxygen to generate reactive oxygen species which, in turn, induce cellular toxicity. In some embodiments, the nanoparticles of the present invention may comprise photosensitizers in order to employ PDT. PDT was initially applied clinically to cutaneous and bladder malignancy because those tumors can easily be exposed to light due to their location. While brain tumors cannot be exposed to light as easily as superficial tumors, even the deepest brain tumors can be easily illuminated after traditional surgical exposure.

In some embodiments, the present invention provides the following major benefits in PDT: 1. NP can specifically deliver photosensitizers to tumor cells via tumor specific ligands; 2. NP allows the photosensitizers to be encapsulated and delivered at a high concentration; 3. The NP matrix (e.g. hydrogel) provides protection, for the active agents, from enzymatic or environmental degradation; 4. The NP matrix can reduce immunogenicity and other side effects. In some embodiments, a photosensitizer that can produce a high amount of singlet oxygen but has a low in vivo PDT efficacy because of enzymatic interference, can become an efficient PDT agent by being loaded into/onto nanoparticles of the present invention. In one exemplary embodiment of the present invention, methylene blue, a blue colored, fluorescent dye and an efficient singlet oxygen producer, is often reduced into a colorless, non-fluorescent and photodynamically inactive form (neutral leuko-methylene blue) by plasma enzymes, when applied systemically in vivo. However, inactivation of free methylene blue molecules could be largely prevented by encapsulation in NPs of the present invention.

In some embodiments, the present invention provides compositions, methods, kits, reagents, and systems for the delineation of tumor tissue. Administration of dye-loaded nanoparticles of the present invention induces staining of tumor tissue while the surrounding non-cancerous tissue remains unstained (e.g. substantially unstained, completely unstained, profoundly unstained, unstained upon visual inspection, unstained to the naked eye, etc.). In some embodiments, upon administration, dye loaded nanoparticles concentrate in tumor tissue (e.g. >10% of administered particles reside in tumor . . . >20% . . . >30% . . . >40% . . . >50% . . . >60% . . . >70% . . . >80% . . . >90% . . . >95% . . . >99% of administered particles reside in tumor). In some embodiments, dye-loaded nanoparticles do not concentrate in non-cancerous (e.g. non-tumor) tissue (e.g. <50% of administered particles reside in non-tumor tissue . . . <40% . . . <30% . . . <20% . . . <10% . . . <5% . . . <1% of administered particles reside in non-tumor tissue). In some embodiments, following administration of dye-loaded nanoparticles, tumor tissue remains stained (e.g. visually detectable staining) and surrounding non-tumor tissue remains unstained (e.g. visually undetectable staining, clearly contrastable to tumor) for an extended time period (e.g. 5 minutes . . . 10 minutes . . . 30 minutes . . . 1 hour . . . 2 hours . . . 3 hours . . . 4 hours . . . 6 hours . . . 8 hours . . . 10 hours . . . 12 hours . . . 24 hours . . . or longer). In some embodiments, administration of dye-loaded nanoparticles provides visual contrast between tumor and non-tumor tissue for sufficient time to perform surgical resection (e.g. 30 minutes . . . 1 hour . . . 2 hours . . . 3 hours . . . 4 hours . . . 6 hours . . . 8 hours . . . 10 hours . . . 12 hours).

In some embodiments, dye-loaded nanoparticles comprise a dye component (e.g. Coomassie blue), one or more nanoparticle components (e.g. polyacrylamide, N-(3-Aminopropyl)methacrylamide, organically-modified silicate, etc.), optionally one or more targeting moieties (e.g. tumor-specific peptides (e.g. F3, Chlorotoxin, etc.), and optionally one or more additional functional moieties (e.g. chemotherapeutic, fluorescent dye, etc.). In some embodiments, the components of a dye-loaded nanoparticle are selected to provide desired targeting, delineation, contrast, kinetics, and stability characteristics. In some embodiments, a nanoparticle comprises a dye-loaded nanoparticle without a targeting moiety. In some embodiments, a nanoparticle comprises a dye-loaded nanoparticle with a tumor-specific targeting moiety (e.g. tumor-specific peptide). In some embodiments, a nanoparticle comprises a dye-loaded nanoparticle with a plurality of different targeting moieties (e.g. one or more tumor-specific moieties, cancer specific moieties, etc.). In some embodiments, nanoparticle components (e.g. polyacrylamide, N-(3-Aminopropyl)methacrylamide, organically-modified silicate, those listed herein, etc.) are selected to provide a nanoparticle with the desired size, solubility, viscosity, stability, loadability, etc.

### EXPERIMENTAL

### Example 1

### Nanoparticle Synthesis

During development of embodiments of the present invention, Coomassie Blue and Indocyanine green loaded polyacrylamide (PAA) nanoparticles were synthesized. Amine-functionalized PAA nanoparticles were synthesized by a microemulsion method. An aqueous solution containing acrylamide, 3-(aminopropyl)methacrylamide hydrochloride salt, and methylene-bis-acrylamide is mixed with hexane solution containing Brij 30 and AOT. Nanoparticle synthesis was initiated by ammoninum persulfate and TEMED. After overnight reaction at room temperature, the nanoparticle solution was evaporated and the resultant thick residue was taken in ethanol. A thorough wash with ethanol and water followed by freeze-drying produced a fine powder of nanoparticles. The Coomassie Blue or Indocyanine green was post-loaded into the blank particles at room temperature by adding dyes in DMSO into the NP suspension in PBS, stirring the mixture for 2 hours and washing with water and PBS multiple times.

### Example 2

### Synthesis of Peptide-Targeted Dye-Loaded PAA Nanoparticles

The peptide conjugation to surface amines of the nanoparticle surface was made through a bifunctional conjugating ligand, sulfo-SMCC. The nanoparticle suspension in PBS (pH 7.4) was mixed with sulfo-SMCC and stirred at room temperature for 2 h and then subjected to thorough washing to remove any unreacted ligands. The collected nanoparticle solution was treated with wild type (AKVKDEPQRRSARLSAKPAPPKPEPKPKKAPAKK), scrambled (AVKPKAARALPSQRSRPPEKAKKPPDKPAPEKKK) F3-peptides with cysteine (F3-Cys; 32-amino acids) or HIV Tat peptide and gently stirred overnight at room temperature. The reaction mixture was then treated with L-Cysteine for 2 h in order to scavenge any unreacted ligand and to minimize the potential for non-specific binding. The resultant nanoparticle solution was thoroughly washed with water and PBS. The absorbance of nanoparticle solutions was measured to evaluate dye content.

### Example 3

### In Vivo Administration

In an exemplary in vivo administration of the present invention, 300 g Sprague Dauley rats were used to create a model to evaluate tumor delineation in the CNS by dye-loaded nanoparticles. Rats were anesthetized and a section of both parital bones were removed. Subsequently, the dura was removed within the borders of parietal bone defect. 10,000 9 L gliosarcoma cells (grown in culture) were injected into the upper right corner of the window created in the parietal bone. A glass cover slip was fixed to the surface of the remaining skull bones with super glue to form a water tight seal. Tumor growth was monitored visually. When a visible tumor was apparent, and while adjacent brain was also apparent, an animal was deemed ready for use in evaluating delineation by nanoparticles. Nanoparticles were administered at a dose of 500 mg/kg body weight via a cannula in a femoral vein over the course of 5 minutes. Staining of the tumor region was evident by visual inspection. Clear delineation between the tumor and non-tumor tissue was observable. The boundaries of the tumor were clearly evident. Upon nanoparticle administration, the color of the tumor darkens and the color of the brain becomes more pale creating conditions that are optimal for tumor delineation. For non-targeted nanoparticles, color change peaks at 20-30 minutes and diminishes significantly by 60 minutes. F3-targeted nanoparticles cause persistent delineation of tumor that does not diminish during the hour of video recording. A quantitative evaluation of color change within the tumor (judged by image J analysis of hue within specific regions of interest) is demonstrated in the table below. Because they are visually similar before treatment, the hue of the tumor and the brain are nearly identical. After treatment there is a profound difference in the measured hue between normal brain and tumor tissue.

### Example 4

### In Vivo Administration of Dye-Loaded Nanoparticles

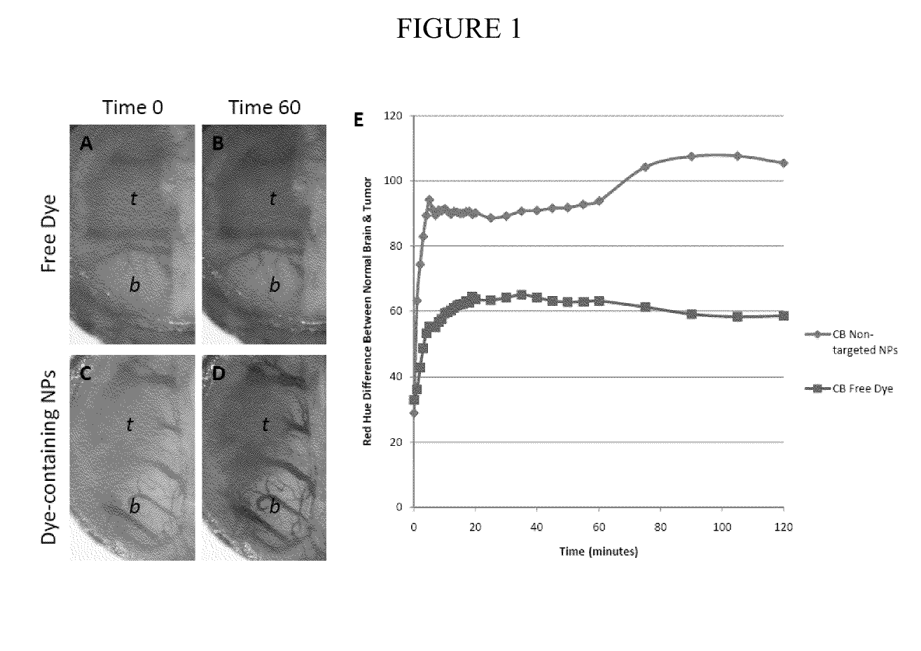

In vivo studies performed during development of embodiments of the present invention demonstrate the application of dye-containing nanoparticles to brain tumor delineation. A brain tumor window model provides visualization of tumor adjacent to normal, non-cancerous brain in real time in a living rat. This model provides conditions encountered in the operating room during a brain tumor resection. Specifically it recapitulates the difficulty of visually delineating cancerous and non-cancerous brain tissue (SEE FIGS. 1A and 1C). After administration of dye or dye loaded-nanoparticles, tumor delineation is greatly enhanced (SEE FIGS. 1B and 1D). Dye-containing nanoparticles provide more intense delineation than free dye (SEE FIG. 1E). The degree of visible delineation between cancerous and non-cancerous brain tissue is more than adequate to guide a brain tumor resection that would be significantly more complete than a traditional resection. MRI and histologic analysis confirms that the area delineated by nanoparticles correlates with tumor margins. Furthermore, because tumor delineation by nanoparticles is more intense than with free dye, a lower dose of nanoparticles is required to provide adequate visual delineation. By using a lower dose of contrast agent, the chances of significant toxicity from administration are diminished. In some embodiments, a single dose of nanoparticles adequately delineates tumor margins for the duration of a brain tumor resection (e.g. 2-4 hours, 6 hours, etc.). To achieve long-lasting delineation with free dye, multiple or continuous dosage can be necessary. Re-dosing a contrast agent during tumor resection can create artifactual tissue staining due to the leakiness of blood vessels both inside and outside of the tumor that are normally disrupted during brain tumor surgery.

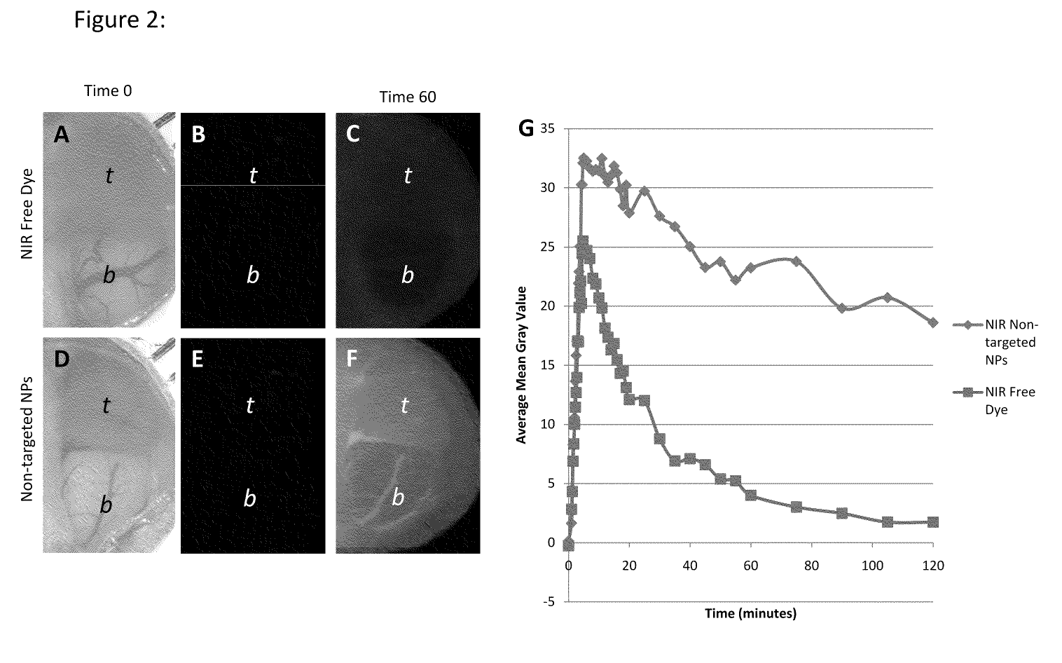

Experiments were performed during development of embodiments of the invention to define the kinetics of nanoparticle-enabled tumor delineation. The brain tumor window offers a realistic model of the clinical challenge of distinguishing lesion margins during brain tumor resection. With respect to measuring the degree and persistence of delineation, it is ideal to eliminate the background appearance of the tissues in the preparation so that the location of nanoparticles can be evaluated directly. Near-infrared imaging technology was employed to allow tracking of nanoparticles in the brain tumor window system. At baseline there is no perceptible emission of living tissue in the near-IR range of the spectrum (SEE FIGS. 2B and 2E) and, therefore, no visual difference between normal brain tissue and cancerous tissue. After administration, dye or dye-containing nanoparticles appear within the blood vessels and are concentrated within the tumor (SEE FIGS. 2C and 2F). Because the tissue has no intrinsic emission in the IR-range, the brightness of the tissue reflects the presence of dye or dye-containing nanoparticles. Serial evaluation of brain tumor window animals using IR imaging indicates that dye-containing nanoparticles create significantly more intense and longer-lasting tumor delineation than free dye.

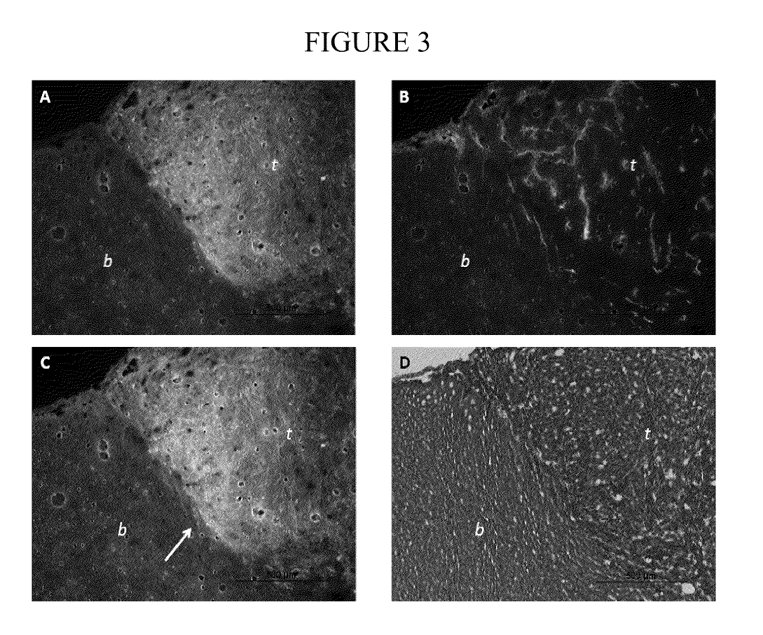

The distribution of dye-containing nanoparticles was evaluated on a tissue level. A tumor model system that expresses green fluorescent protein (GFP) and rhodamine-loaded nanoparticles was created. The co-localization of rhodamine-containing nanoparticles in to GFP-expressing brain tumors confirms that intravenously injected nanoparticles are taken up by brain tumors (SEE FIG. 3). The nanoparticles are found in a pattern characteristic of tumor blood vessels (SEE FIG. 3C), indicating that they are closely associated with tumor vasculature and possibly the perivascular tissue. Even at the tumor margins where tumor cells are sparse, nanoparticles can be found closely associated with angiogenic vessels (SEE FIG. 3D).

We claim:

1. A method for delineation of tumor boundaries, comprising:
a. administering a nanoparticle-based optical dye to a subject, wherein said subject has a tumor; and
b. observing said nanoparticle-based optical dye localized in said tumor.

2. The method of claim 1, further comprising a step between step a) and b) of localizing of said nanoparticle-based optical dye in said tumor.

3. The method of claim 1, wherein said method further comprises the step of:
c. resecting said tumor using said optical dye to select resection boundaries.

4. The method of claim 1, wherein said nanoparticle-based optical dye is targeted to said tumor with a tumor-specific targeting moiety.

5. The method of claim 4, wherein said tumor-specific targeting moiety is a tumor-specific peptide.

6. The method of claim 5, wherein said tumor-specific targeting peptide comprises F3.

7. The method of claim 1, wherein said nanoparticle comprises polyacrylamide.

8. The method of claim 1, wherein said optical dye is selected from the group of Coomassie Blue, Methylene Blue, and or Indocyanine Green.

9. The method of claim 1, wherein said tumor arises from cancer, selected from the list of brain cancer, bladder cancer, melanoma, breast cancer, colon cancer, rectal cancer, pancreatic cancer, endometrial cancer, prostate cancer, kidney (renal cell) cancer, skin cancer (nonmelanoma), thyroid cancer, and lung cancer.

10. The method of claim 1, wherein said nanoparticle-based optical dye remains localized within said tumor for at least 2 hours.

11. The method of claim 1, wherein said delineation is observable with the naked eye.

12. A nanoparticle-based optical dye composition comprising a nanoparticle component and an optical dye component, wherein said optical dye component is loaded into said nanoparticle component.

13. The nanoparticle-based optical dye composition of claim 12, wherein said nanoparticle component comprises polyacrylamide.

14. The nanoparticle-based optical dye composition of claim 12, wherein said nanoparticle component is selected from the group of Coomassie Blue, Methylene Blue, and Indocyanine Green.

15. The nanoparticle-based optical dye composition of claim 12, further comprising a tumor-specific targeting moiety.

16. The nanoparticle-based optical dye composition of claim 15, wherein said tumor-specific targeting moiety comprises a tumor-specific peptide.

17. The nanoparticle-based optical dye composition of claim 16, wherein said tumor-specific targeting peptide comprises F3.

18. The nanoparticle-based optical dye composition of claim 16, wherein said tumor-specific targeting peptide comprises Chlorotoxin.

19. The nanoparticle-based optical dye composition of claim 12, wherein said nanoparticle-based optical dye composition is stable for at least 2 hours under physiological conditions.
